(19) United States
(12) Patent Application Publication
(10) Pub. No.: US 2010/0274185 A1
(43) Pub. Date: Oct. 28, 2010

(54) AUTOMATIC INJECTION SYRINGE ASSEMBLY

(76) Inventor: Thomas Chun, Northridge, CA (US)

Correspondence Address: ITALIA IP, 3500 WEST OLIVE AVE., SUITE 300, BURBANK, CA, 91505, US

(21) Appl. No.: 12/431,144

(22) Filed: Apr. 28, 2009

(51) Int. Cl. A61M 5/31; A61M 5/20; A61M 5/32

(52) U.S. Cl. 604/89; 604/135

(57) ABSTRACT

An apparatus for actuating a syringe. The syringe includes a barrel, a plunger and a needle. A needle shield is near the front of the barrel and is movable from a first position enclosing the needle and a retracted position wherein the needle is exposed. A spring abuts the plunger. A cap has an interior wall for engaging the spring and releasably engages the plunger. The spring is compressed between the plunger and the interior wall when the cap releasably engages the plunger. A resilient member releasably secures the cap in releasable engagement with the plunger and is movable from a first position to a compressed position when the needle shield is moved from its first position to its retracted position. The cap disengages the plunger when the resilient member is moved to the compressed position wherein the spring expands to provide a medicament delivery pressure to the plunger.

### BACKGROUND OF INVENTION

This application relates generally to an automatic injection syringe assembly. More specifically, this application relates to an automatic injection syringe assembly that allows a medicament to be introduced into the body through the skin.

### SUMMARY

Providing injections of medicaments to a patient is a highly successful mode of treatment. However, using a typical syringe often requires great skill and care so as to avoid injecting too deep, scratching the patient with the needle, and the like. Moreover, having the patient inject himself or herself provides the same challenges in addition to the general nervousness or anxiety frequently involved with injecting oneself. The present application discloses a automatic injection syringe assembly that decreases the skill and steady-hand that injections typically require.

In particular, this application discloses a syringe assembly comprising: a barrel having a forward end and a rear end and defining a reservoir within which a medicament may be contained; a hollow needle coupled to the barrel forward end and in fluid communication with the reservoir; a plunger having a first end with a stopper positioned in the reservoir and a pad for receiving medicament delivery pressure for causing the plunger to move within the reservoir to expel the medicament from the reservoir; a needle shield positioned near the forward end of the barrel, the needle shield movable from a first position enclosing the needle and a retracted position wherein the needle is exposed; a spring having a first end and a second end, wherein the spring first end engages the plunger pad; a cap having an interior wall for engaging the second end of the spring and operable to releasably engage the plunger, wherein the spring is compressed between the plunger pad and the interior wall when the cap is releasably engaged with the plunger; and a resilient member operable to releasably secure the cap in releasable engagement with the plunger, wherein the resilient member is movable from a first position to a compressed position when the needle shield is correspondingly moved from the needle shield first position to the needle shield retracted position, wherein the cap disengages the plunger when the resilient member is moved to the compressed position such that the spring is operable to expand and thereby provide a medicament delivery pressure to the plunger pad.

The present application also discloses an apparatus for applying a medicament delivery pressure to a plunger pad of a plunger of a syringe, the apparatus comprising: a spring having a first end and a second end, wherein the spring first end is operable to engage the plunger pad; a cap having an interior wall for engaging the second end of the spring and operable to releasably engage the plunger, wherein the spring is compressed between the plunger pad and the interior wall when the cap is releasably engaged with the plunger; and a resilient member operable to releasably secure the cap in releasable engagement with the plunger, wherein the resilient member is movable from a first position to a compressed position, and wherein the cap disengages the plunger when the resilient member is moved to the compressed position such that the spring is operable to expand and thereby provide a medicament delivery pressure to the plunger pad.

The present application further discloses an apparatus for actuating a syringe, the syringe comprising a barrel, a plunger and a needle, the apparatus comprising: a needle shield positioned near a forward end of the barrel, the needle shield movable from a first position enclosing the needle and a retracted position wherein the needle is exposed; a spring having a first end and a second end, wherein the spring first end abuts the plunger; a cap having an interior wall for engaging the second end of the spring and operable to releasably engage the plunger, wherein the spring is compressed between the plunger and the interior wall when the cap is releasably engaged with the plunger; and a resilient member operable to releasably secure the cap in releasable engagement with the plunger, wherein the resilient member is movable from a first position to a compressed position when the needle shield is correspondingly moved from the needle shield first position to the needle shield retracted position, wherein the cap disengages the plunger when the resilient member is moved to the compressed position such that the spring is operable to expand and thereby provide a medicament delivery pressure to the plunger.

### BRIEF DESCRIPTION OF THE DRAWINGS

The drawings, when considered in connection with the following description, are presented for the purpose of facilitating an understanding of the subject matter sought to be protected.

FIG. 1 is an exploded view of a first embodiment of a syringe assembly as disclosed herein;

FIG. 2 is a cross-sectional view of the syringe assembly of FIG. 1 showing the syringe assembly prior to an injection;

FIG. 3 is a cross-sectional view of the syringe assembly of FIG. 1 showing the syringe assembly during an injection; and

FIG. 4 is a partial cross-sectional view of a second embodiment of a syringe assembly as disclosed herein.

### DETAILED DESCRIPTION

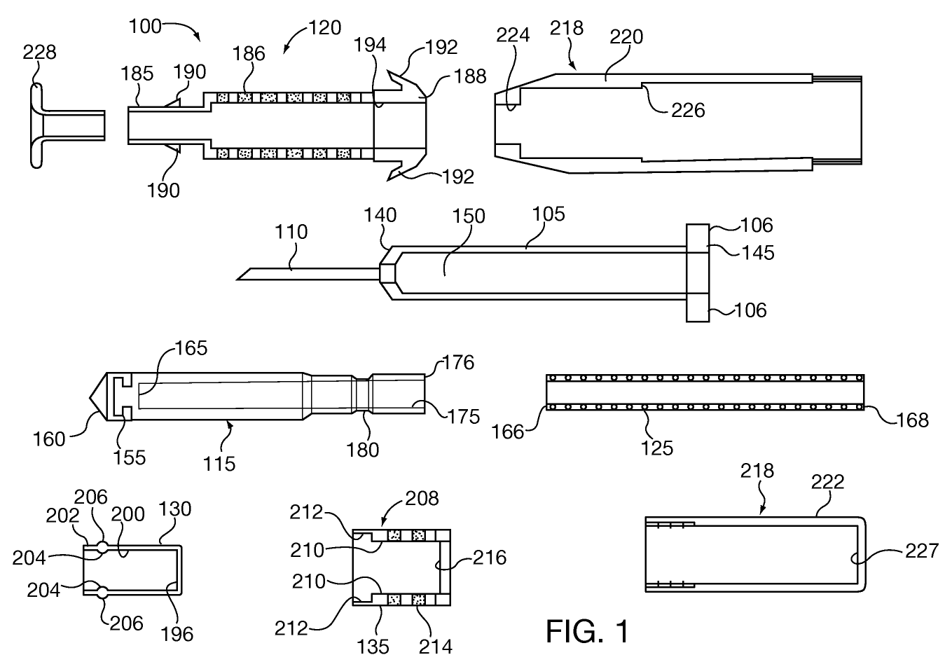

Referring now to FIGS. 1-3, an illustrative embodiment of a syringe assembly 100 is shown. The syringe assembly 100 generally includes a barrel 105, a hollow needle 110, a plunger 115, a needle shield 120, a spring 125, a cap 130 and a resilient member 135.

The barrel 105 has a forward end 140, a rear end 145 and a reservoir 150 for containing a medicament. In an alternative embodiment, as described in U.S. patent application Ser. No. 12/429,985, entitled “Syringe Having Extended Blending Path” by Thomas Chun, co-filed with the present application and herein incorporated by reference, the reservoir 150 may be divided into a solvent containing portion and a solute containing portion by a barrier where the barrier is selectively openable to provide fluid communication between the solute containing portion and solvent containing portion; further, a flow path member may be disposed between the reservoir 150 and needle 110 such that the flow path member establishes a flow path along which a medicament solution generated by blending the solvent and solute may flow in response to movement by the plunger 115. In yet another alternative embodiment, a flow path member, as disclosed in the previously mentioned “Syringe Having Extended Blending Path” to Thomas Chun, may be disposed in a secondary barrel coupled to the barrel forward end 140 such that the secondary barrel and barrel 105 are in fluid communication whereby the medicament is blended in the secondary barrel.

The hollow needle 110 is coupled to the forward end 140 of the barrel 105 via any suitable means and is in fluid communication with the reservoir 150. In addition, the needle 110 may either be releasably or rigidly secured to the forward end 140 of the barrel 105. The needle 110 may be of any suitable length or gage for injecting a medicament into a patient. The needle 110 may be configured for subcutaneous injection, intramuscular injection, intravenous injection, or the like.

The plunger 115 has a first end 155 with a stopper 160 for sliding engagement within the reservoir 150. The plunger 115 also includes a pad 165 for receiving medicament delivery pressure from the spring 125 whereby medicament may be expelled from the reservoir 150 through the needle 110. In the illustrative embodiment, the pad 165 is disposed in at the bottom of bore 175 in the second end 176 of the plunger 115. The pad 165 is operable to engage a first end 166 of the spring 125 whereby the spring 125 is operable to provide a medicament delivery pressure thereto. The bore 175 may also be adapted to receive a portion of the spring 125 therein. Additionally, as will be discussed below, the circumference of the plunger may include an annular groove 180 for releasably receiving one or more inward projections 204 of one or more deflectable flanges 202 of the cap 130.

The needle shield 120 is positioned the near the forward end 140 of the barrel 105. The needle shield 120 includes a shield portion 185, a spring body 186 and a rear portion 188. The needle shield 120 is movable from a first position where the shield portion 185 encloses the needle 110 and a retracted position where the needle 110 is exposed. Movement of the needle shield 120 may be accomplished by pressing the shield portion 185 against the skin of a patient. The spring portion 185 may serve to bias the shield portion 185 in the first position and cause shield portion 185 to return to the first position when removed from the patient's skin. Also, in one embodiment, the shield portion 185 may include one or more tangs 190 for engaging the front end 224 of a casing 218 whereby the tangs 190 prevent the shield portion 185 from fully entering the casing 218 when the shield portion 185 is moved to the retracted position. In one embodiment, when the shield portion 185 is moved to the retracted position, the shield portion 185 is at least partially encased by the casing 218. The needle shield 120 is generally tubular in shape with a central aperture 194 for receiving at least a portion of the barrel 105 therein. The rear portion 188 may securedly engage the forward end 140 of the barrel 105 such that, as will be discussed further below, movement of the needle shield 120 to a retracted position may cause the barrel 105 to move slightly reward whereby the flanges 106 of the barrel 105 are operable to compress the resilient member 135. In addition, in one embodiment, the rear portion 188 may include one or more deflectable flanges 192 disposed thereabouts and operable to expand and engage an interior shoulder 226 of the casing 218 as the needle shield 120 is moved reward within the casing 218 after the needle shield 120 has been moved to a retracted position. As will be discussed further below, the deflectable flanges 192 engage the shoulder 226 when the medicament is being delivered so as to stabilize the barrel 105 and needle 110 therewhile.

The spring 125 includes a first end 166 and a second end 168. As will be discussed further below, the spring 125 is operable to be compressed between the interior wall 196 of the cap 130 and the plunger pad 165, and, upon release of the cap 130 from the resilient member 135, the spring 125 is operable to provide a medicament delivery pressure to the plunger pad whereby medicament is expelled through the needle 110. In one embodiment, the spring 125 is also operable to move the barrel 105 and needle 110 forward such that the needle 110 may pierce the skin of the patient prior to the spring 125 delivering the medicament delivery pressure to the plunger pad 165. In one embodiment, the spring 125 is a compression spring, although, as will be appreciated by those skilled in the art, other resilient or expandable mechanism(s) for providing a medicament delivery pressure to the plunger pad 165 may be employed.

The cap 130 includes an interior wall 196 for engaging the second end 168 of the spring 125 such that the spring 125 may be compressed between the plunger pad 165 and the interior wall 196. The cap 130 may be tubular with a central aperture 200. The aperture 200 may be adapted to receive a portion of the spring 125 and a portion of the plunger 115. The cap 130 may be adapted to releasably receive a portion of the plunger 115 such that the spring 125 may remain compressed between the interior wall 196 and plunger pad 165 when the plunger 115 and cap 130 are in such releasable engagement. In one embodiment, the cap 130 includes one or more deflectable flanges 202 having at least one projection 204 projecting inwardly relative to the aperture 200. The inward projection(s) 204 may be adapted to be releasably received by the plunger groove 180. In this arrangement, as will be discussed further below, the resilient member 135 may bias the flange(s) 202 towards the groove 180 such that each inward projection 204 may be releasably received within the groove 180. When each inward projection 204 is retained within the groove 180, the spring 125 is maintained in a compressed state between the interior wall 196 and plunger pad 165. Each flange 202 may also include one or more projections 206 projecting outwardly relative to the aperture 200. As will be discussed further below, each outward projection 206 may be configured to releasably engage a first step 210 within the resilient member 135 when the resilient member is in a first position and, optionally engage a second step 212 within the resilient member 135 when the resilient member 135 is in a compressed position. When each outward projection 206 is engaged with the first step 210, each flange 202 is biased towards the plunger 115 such that each inward projection 204 engages the plunger groove 180, and as each outward projection 206 is engaged with the second step 212 within the resilient member 135, each flange 202 moves away from the plunger 115 whereby each inward projection 204 disengages the plunger groove 180 whereby the spring 125 is permitted to expand and thereby provide a medicament delivery pressure to the plunger pad 165 such that medicament is expelled from the needle 110 into the patient.

The resilient member 135 includes a front portion 208 having a first step 210 and, optionally, a second step 212. The resilient member 135 is generally tubular with an aperture 216 for receiving at least a portion of the cap 130. The resilient member 135 is movable from a first, or extended, position to a compressed position when the needle shield 120 is correspondingly moved from the needle shield first position to the needle shield retracted position. The resilient member 135 may be moved to the compressed position by flanges 106 of the barrel 105 as the barrel 105 is moved slightly rearwardly by movement of the needle shield 120 to a retracted position. Alternatively, the resilient member 135 may be moved to a compressed position by engagement with the needle shield 120 as the needle shield 120 is moved to a retracted position. The first step 210 engages the outward projection(s) 206 of the cap 130 such that cap flange(s) 202 are deflected towards the plunger 115 whereby the inward projection(s) 204 are maintained within the plunger groove 180 and the spring 125 maintained in a compressed position between the plunger pad 165 and interior wall 196 of the cap 130. As the resilient member 135 is moved to a compressed position, the cap flange(s) 202 move away from the plunger 115 such that the inward projection(s) 204 disengage the plunger groove 180 whereby the spring 125 is permitted to expand and thereby provide a medicament delivery pressure to the plunger pad 165. In one embodiment, when the resilient member 135 is moved to the compressed position, the outward projection(s) of the cap 130 engage the second step 212. The resilient member 135 may also include a spring portion 214 that provides resiliency, and/or compressibility, to the resilient member 135. Alternatively, the resilient member 135 may be formed from a compressible material such that the resilient member 135 may move from a first position to a compressed position.

In one embodiment, the assembly includes a casing 218 for encompassing the other components of the assembly 100. The casing 218 includes a front 220 and rear 222 hollow tubular shell, wherein the front tubular shell 220 is open at one end 224 for the shield portion 185 and needle 110 to pass through. The rear shell 222 attaches to the front shell 220 to form a complete shell to retain the internal structure of the syringe assembly and provide a surface to be held by the user. As previously mentioned, the front shell 220 may include an interior shoulder 226 for engaging deflectable flanges 192 of the needle shield 120 when the medicament is being delivered so as to stabilize the barrel 105 and needle 110 therewhile. Also, rear shell 222 may have a rear wall 222 to provide a surface to assist in the compression of the resilient member 135. Additionally, a front cap 228 may be provided to engage the front end 224 of the casing 218 for protecting the needle shield 120 and needle 110 prior to use.

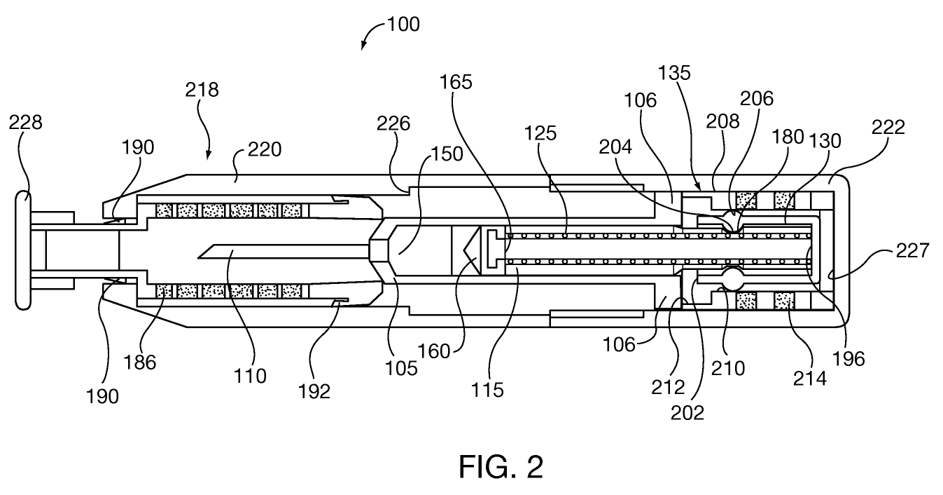

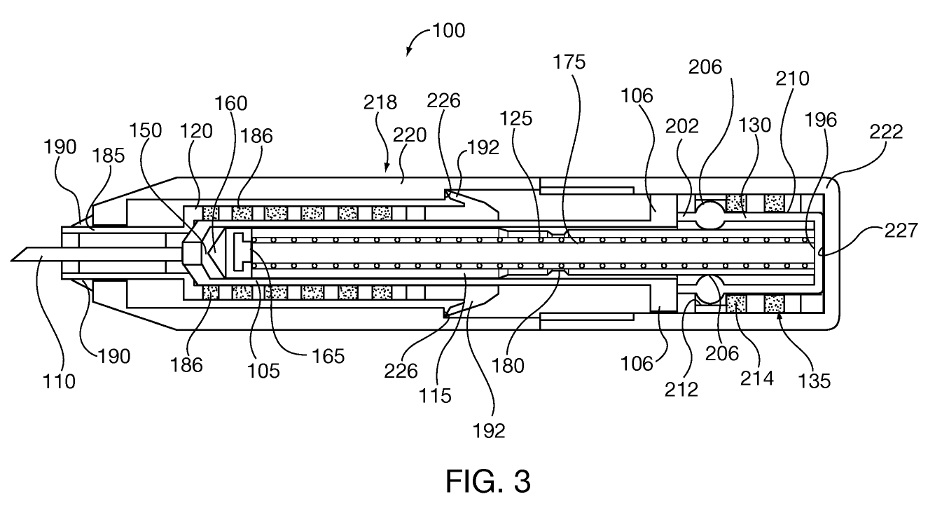

Referring particularly to FIGS. 2 and 3, operation of the syringe assembly 100 will be summarized. First, the front cap 228 may be removed to expose the shield portion 185 of the needle shield 120. The shield portion 185 may then be placed against a patient's skin and pressure applied thereto by pressing the casing 218 toward the patient such that the needle shield 120 is moved to a retracted position. In an alternative embodiment, movement to the retracted position exposes the needle 110. After the needle shield 120 is moved to a retracted position and with further pressure against the patient's skin, the rear portion 188 of the needle shield 120 is moved towards the shoulder 226 of the casing 218 wherein the flanges 192 expand and engage the shoulder 226 whereby the needle shield 120 and barrel 105 become disengaged. In addition, with continued pressure against the patient's skin, the barrel 105 is moved slightly towards the rear wall 222 of the casing 218 and the barrel flanges 106 begin to compress the resilient member 135. As the resilient member 135 is moved to a compressed position, the outward projections 206 of the cap disengage the first step 210 of the resilient member 135 and the inward projections 204 disengage the plunger groove 180 whereby the spring 125 is permitted to expand. Expansion of the spring 125 provides a medicament delivery pressure to the plunger pad 165 to move the plunger 115 towards the barrel forward end 140 so that medicament is expelled from the barrel reservoir 150, though the needle 110 and into the patient. In one embodiment, prior to medicament being expelled, the spring 125 first drives the barrel 105 and needle 110 partially through the apeture 194 of the needle shield 120 such that the needle 110 is moved out from the casing 218 and into the patient and/or moves the needle 110 further into the patient, after which the medicament is expelled from the barrel 105 and needle 110 into the patient.

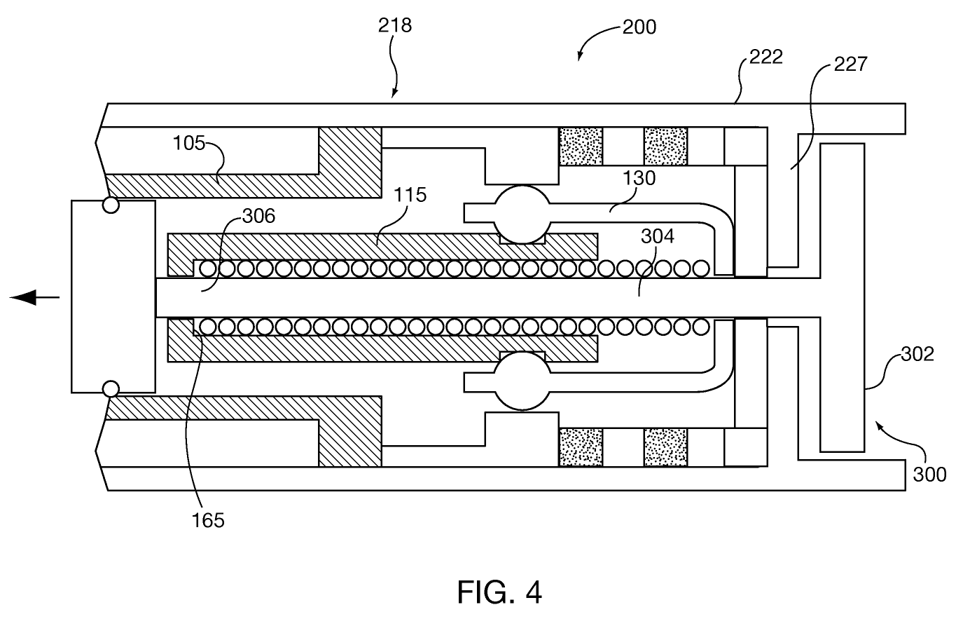

Referring now to FIG. 4, a partial view of an alternative syringe assembly 200 is shown. The syringe assembly 200 is similar to that shown in FIGS. 1-3, so like numerals indicate like elements throughout. In the embodiment of FIG. 4, the assembly 200 includes a second plunger 300 for eliminating air and/or a portion of the medicament in the reservoir to ensure only medicament is injected into the patient. The second plunger 300 is generally activated prior to pressing the syringe assembly against a patient's skin to induce an injection as previously described. The plunger 300 generally includes a shaft 304 having a first end 306 and a second end having a thumb pad 302. The shaft 304 extends through the rear wall 227 of the rear shell 222, the cap 130 and the plunger 115 where the first end 306 engages the stopper 160. The thumb pad 302 is positioned outside of the casing 222. In operation, a user presses the thumb pad 302 towards the rear wall 227 such that the stopper 160 is moved to expel a portion of air and/or medicament from the reservoir of the barrel 105. In one embodiment, the rear wall 227 is operable to limit the movement of the second plunger 300 such that the second plunger 300 is not capable of expelling a substantial amount of medicament from the barrel 115.

While the present disclosure has been described in connection with what is considered the most practical and preferred embodiment, it is understood that this disclosure is not limited to the disclosed embodiments, but is intended to cover various arrangements included within the spirit and scope of the broadest interpretation so as to encompass all such modifications and equivalent arrangements.

What is claimed is:

1. A syringe assembly comprising:
a barrel having a forward end and a rear end and defining a reservoir within which a medicament may be contained;
a hollow needle coupled to the barrel forward end and in fluid communication with the reservoir;
a plunger having a first end with a stopper positioned in the reservoir and a pad for receiving medicament delivery pressure for causing the plunger to move within the reservoir to expel the medicament from the reservoir;
a needle shield positioned near the forward end of the barrel, the needle shield movable from a first position enclosing the needle and a retracted position wherein the needle is exposed;
a spring having a first end and a second end, wherein the spring first end engages the plunger pad;
a cap having an interior wall for engaging the second end of the spring and operable to releasably engage the plunger, wherein the spring is compressed between the plunger pad and the interior wall when the cap is releasably engaged with the plunger; and
a resilient member operable to releasably secure the cap in releasable engagement with the plunger, wherein the resilient member is movable from a first position to a compressed position when the needle shield is correspondingly moved from the needle shield first position to the needle shield retracted position, wherein the cap disengages the plunger when the resilient member is moved to the compressed position such that the spring is operable to expand and thereby provide a medicament delivery pressure to the plunger pad.

2. The syringe assembly of claim 1 further comprising a barrier disposed within the barrel to separate the reservoir into a solvent containing portion and a solute containing portion, wherein the barrier is selectively openable to provide fluid communication between the liquid solvent containing portion and the solid solute containing portion.

3. The syringe assembly of claim 2 further comprising a flow path member disposed between the reservoir and needle, wherein the flow path member establishes a flow path along which a medicament solution generated by blending the solvent and the solute may flow responsively to movement of the plunger under the influence of a medicament delivery pressure.

4. The syringe of claim 1 wherein the plunger includes an annular groove and wherein the cap includes at least one deflectable flange having at least one projection for releasably engaging the plunger groove.

5. The syringe of claim 1 wherein cap includes at least one deflectable flange and a projection extending from the flange, and wherein the resilient member includes a step for releasably engaging the projection and thereby releasably secure the cap in releasable engagement with the plunger.

6. The syringe assembly of claim 1 further comprising a second plunger having a first end and a thumb pad, wherein the first end engages the plunger pad such that pressure on the thumb pad expels a portion of medicament from the reservoir.

7. The syringe of claim 1 wherein the needle shield comprises a spring portion for biasing the needle shield in the first position.

8. The syringe of claim 1 wherein the needle shield engages the resilient member when the needle shield is moved to the retracted position.

9. The syringe of claim 1 further comprising a casing for encompassing the barrel, the needle, the plunger, the needle shield, the spring, the cap and the resilient member, the casing including an open end wherein the needle shield extends at least partially through the open end when the needle shield is in the first position and wherein the needle extends at least partially through the open end when the needle shield is moved to the retracted position.

10. The syringe of claim 1 wherein the needle shield returns to the first position after the medicament has been expelled from the reservoir.

11. An apparatus for applying a medicament delivery pressure to a plunger pad of a plunger of a syringe, the apparatus comprising:
a spring having a first end and a second end, wherein the spring first end is operable to engage the plunger pad;
a cap having an interior wall for engaging the second end of the spring and operable to releasably engage the plunger, wherein the spring is compressed between the plunger pad and the interior wall when the cap is releasably engaged with the plunger; and
a resilient member operable to releasably secure the cap in releasable engagement with the plunger, wherein the resilient member is movable from a first position to a compressed position, and wherein the cap disengages the plunger when the resilient member is moved to the compressed position such that the spring is operable to expand and thereby provide a medicament delivery pressure to the plunger pad.

12. The apparatus of claim 11 wherein the cap includes at least one deflectable flange having at least one projection for releasably engaging the plunger.

13. The apparatus of claim 11 wherein cap includes at least one deflectable flange and a projection extending from the flange, and wherein the resilient member includes a step for releasably engaging the projection and thereby releasably secure the cap in releasable engagement with the plunger.

14. The apparatus of claim 11 wherein the spring is a compression spring.

15. An apparatus for actuating a syringe, the syringe comprising a barrel, a plunger and a needle, the apparatus comprising:
a needle shield positioned near a forward end of the barrel, the needle shield movable from a first position enclosing the needle and a retracted position wherein the needle is exposed;
a spring having a first end and a second end, wherein the spring first end abuts the plunger;
a cap having an interior wall for engaging the second end of the spring and operable to releasably engage the plunger, wherein the spring is compressed between the plunger and the interior wall when the cap is releasably engaged with the plunger; and
a resilient member operable to releasably secure the cap in releasable engagement with the plunger, wherein the resilient member is movable from a first position to a compressed position when the needle shield is correspondingly moved from the needle shield first position to the needle shield retracted position, wherein the cap disengages the plunger when the resilient member is moved to the compressed position such that the spring is operable to expand and thereby provide a medicament delivery pressure to the plunger.

16. The apparatus of claim 15 wherein the cap includes at least one deflectable flange having at least one projection for releasably engaging the plunger.

17. The apparatus of claim 15 wherein cap includes at least one deflectable flange and a projection extending from the flange, and wherein the resilient member includes a step for releasably engaging the projection and thereby releasably secure the cap in releasable engagement with the plunger.

18. The apparatus of claim 15 wherein the spring is a compression spring.

19. The apparatus of claim 15 wherein the needle shield comprises a spring portion for biasing the needle shield in the first position.

20. The apparatus of claim 15 wherein the needle shield engages the resilient member when the needle shield is moved to the retracted position.
